(19) United States
(12) Patent Application Publication
(10) Pub. No.: US 2011/0004098 A1
(43) Pub. Date: Jan. 6, 2011

(54) ESTER IMAGING AGENTS

(76) Inventors: Antonios Danikas, Amersham (GB); Morten Eriksen, Oslo (NO)

Correspondence Address: GE HEALTHCARE, INC., IP DEPARTMENT 101 CARNEGIE CENTER, PRINCETON, NJ, 08540-6231, US

(21) Appl. No.: 12/919,032

(22) PCT Filed: Mar. 3, 2009
(86) PCT No.: PCT/EP2009/052493

§ 371 Date: Aug. 24, 2010

(30) Foreign Application Priority Data
Mar. 6, 2008 (GB) 0804190.7

(51) Int. Cl. A61B 6/00; A61K 49/00

(52) U.S. Cl. 600/435; 424/9.6; 600/431

(57) ABSTRACT

The present invention relates to contrast agents for optical imaging of oesophageal cancer and Barrett's oesophagus in patients. The contrast agents are esters of 5-aminolevulinic acid (5-ALA). They are useful in the diagnosis of oesophageal cancer and Barrett's oesophagus, for follow up of progress in disease development, and for follow up of treatment of oesophageal cancer and Barrett's oesophagus. The contrast agents are delivered by particular routes of administration.

### FIELD OF THE INVENTION

The present invention relates to contrast agents for optical imaging of oesophageal cancer and Barrett's oesophagus in patients. The contrast agents may be used in the diagnosis of oesophageal cancer and Barrett's oesophagus, for follow up of progress in disease development, and for follow up of treatment of oesophageal cancer and Barrett's oesophagus. The agents are delivered by particular routes of administration.

The present invention also provides new methods of optical imaging of oesophageal cancer and Barrett's oesophagus in patients, for diagnosis and for follow up of disease development and treatment of oesophageal cancer and Barrett's oesophagus.

### BACKGROUND TO THE INVENTION

Oesophageal cancer represents less than 5% of all reported cancer cases, but ca. 30,000 new such cases are diagnosed per annum in the USA and the survival rate is low (see below). Oesophageal cancer can be divided into two major types, squamous cell carcinoma and adenocarcinoma, depending on the type of cells that are malignant. Barrett's oesophagus is a pre-malignant condition which is associated with an increased risk of development of oesophageal cancer; especially adenocarcinoma [Kiesslich et al, Clin. Gastroenterol. Hepatol., 4, 979-987 (2006)]. Chronic reflux increases risk for Barrett's oesophagus, and it has therefore been suggested that gastro oesophageal reflux (GERD) is a risk factor for oesophageal cancer.

Adenocarcinoma of the oesophagus is more prevalent than squamous cell carcinoma in the USA and Western Europe. Oesophageal cancer can be a treatable disease but is rarely curable. The overall 5-year survival rate is between 5% and 30%. Early diagnosis of oesophageal cancer improves the survival rate of the patient. Primary treatment includes surgery alone or chemotherapy in combination with radiation. Chemotherapy used in treatment of oesophageal cancer includes 5-fluorouracil and cisplatin. Lack of precise pre-operative staging is a major clinical problem.

The presence of low grade dysplasia (i.e. abnormal tissue growth) in Barrett's oesophagus is a risk factor for the development of oesophageal cancer, but surveillance currently relies on histopathology [Lim et al, Endoscopy, 39, 581-7 (2007)]. Diagnosis of dysplasia in Barrett's oesophagus is currently via random four-quadrant biopsies every 1 to 2 cm (the Seattle protocol), which is time-consuming and costly [DaCosta et al, Best Pract. Res. Clin. Gastroenterol., 20(1), 41-57 (2006)]. Dysplasia in Barrett's oesophagus is not normally visible during routine endoscopy [Endlicher et al, Gut, 48, 314-319 (2001)].

U.S. Pat. No. 6,035,229 (Washington Research Foundation) describes a system for detecting Barrett's oesophagus utilizing an illumination and imaging probe at the end of a catheter. The document does not disclose an optical contrast agent.

Staining of Barrett's oesophagus tissue in vivo has been compared with staining of biopsy samples in vitro, using the dye methylene blue in the detection of highly dysplastic or malignant tissue [Canto et al, Endoscopy, 33, 391-400 (2001)].

Kiesslich et al [Clin. Gastroenterol. Hepatol., 4, 979-987 (2006)] reported on the use of fluorescein to aid the detection of Barrett's epithelium and associated neoplasia using confocal laser endomicroscopy.

WO 2005/058371 discloses optical imaging contrast agents for imaging of oesophageal cancer and Barrett's oesophagus in vivo. The contrast agents have an affinity for a biological a target which is abnormally expressed in Barrett's oesophagus. The contrast agents of WO 2005/058371 are preferably of formula:

V-L-R
where:
V is one or more vector moieties having affinity for an abnormally expressed target in oesophageal cancer or Barrett's oesophagus;
L is a linker moiety or a bond; and
R is one or more reporter moieties detectable in optical imaging.

A wide range of targets is described, but the target is preferably selected from E-cadherin, CD44, P62/c-myc (HGF receptor), p53 and EGFR/erB-2. The vector (V) is stated to be preferably selected from peptides, peptoid moieties, oligonucleotides, oligosaccharides, fat-related compounds and traditional organic drug-like small molecules. The reporter (R) is preferably a dye that interacts with light in the wavelength region from the ultraviolet to the near-infrared part of the electromagnetic spectrum.

5-amino levulinate (5-ALA) is an intermediate component of the heme biosynthesis pathway which is already present in the majority of human cells [Messmann, Endosc. Clin. N. Amer. 10, 497-512 (2000)]. The major steps in the heme pathway include: the synthesis of 5-ALA; its conversion to protoporphyrin IX (PPIX), and the subsequent addition of iron and conversion to heme. Exogenous administration of 5-ALA is known to result in overproduction and accumulation of PPIX in bladder cancer cells, but not in the normal epithelium. Upon stimulation with blue light, PPIX emits strong fluorescence in the red area. The reasons behind the selective accumulation of PPIX in bladder tumour and not normal cells are not fully understood, but altered activity in three areas may be responsible: higher 5-ALA uptake; increased PPIX production; or decreased PPIX conversion to heme by ferrochelatase [Collaud et al., Curr. Med. Chem. Anticancer Agents, 4, 301-316 (2004)].

5-Aminolevulinic acid (5-ALA) has also been used for photodynamic therapy (PDT) of Barrett's oesophagus, in which 5-ALA administration (typically 50-60 mg/kg) is followed by illumination of the target tissue with bright light. The high levels of PPIX, a potent photosensitiser, induce tissue damage. This technique has been used frequently for the ablation of metaplastic, dysplastic and adenocarcinoma tissue. At these doses PPIX fluorescence cannot, however, be used to differentiate between metaplastic, dysplastic or carcinoma tissues with high specificity [Barr, Gastrointest. Endosc. Clin. N. Am., 10, 421-437 (2000)].

5-ALA has also been used in the endoscopic fluorescence detection of low and high grade dysplasia in Barrett's oesophagus [Endlicher et al, Gut, 48, 314-319 (2001)]. Brand et al [Gastroint. Endosc., 56(4), 479-487 (2002)] describe the detection of high grade dysplasia in Barrett's oesophagus by detection of PPIX fluorescence following oral administration of 5-ALA. Stepinac et al [Endoscopy, 35(8), 663-668 (2003)] disclose that endoscopic fluorescence detection using oral 5-ALA was able to detect high grade neoplasia (new and abnormal tissue growth), in Barrett's oesophagus. Histological grading of the fluorescence positive tissue allowed the calculation of sensitivity and specificity for each study.

A general finding of the Endlicher/Brand/Stepinac 5-ALA studies was the high sensitivity of dysplasia detection with doses of at least 10 mg/kg. However, increasing doses of 5-ALA were accompanied by a decrease in specificity (increasing number of fluorescent-positive, but non-dysplastic tissue). This has been mainly attributed to the presence of inflammation in the examined tissue. False positives were also created by non-dysplastic Barrett's epithelium, and one study reported that 31% of fluorescence positive samples were from non-inflamed metaplasias. The above results suggest that 5-ALA endoscopy can assist in identifying highly dysplastic areas within the Barrett's epithelium, with inflammation being the main factor behind false positives, followed by background from metaplasia (an abnormal change in the nature of the tissue).

U.S. Pat. No. 5,211,938 discloses methods of detecting malignant and non-malignant tissue abnormalities in patients via administration of a PPIX precursor, preferably 5-ALA.

US 2003/0158258 A1 discloses solutions of 5-ALA esters at concentrations suitable for use in diagnosis of PPIX fluorescence in vivo.

WO 96/28412 discloses 5-ALA esters for use in photochemotherapy or in diagnosis. No particular disease states are taught for the diagnostic applications of WO 96/28412.

Collaud et al [J. Control. Rel., 123, 203-210 (2007)] investigated bioadhesive hydrogel formulations for the feasibility of delivery of hexylaminolevulinate (HAL) to Barrett's oesophagus via adhesion to the oesophageal wall. They incorporated a blue dye (patent blue) within each formulation, and used the blue colouration as an indication of the coverage and adhesion of each hydrogel—including studies in normal human volunteers. Imaging based on PPIX or in Barrett's oesophagus patients was not described, but the study was introduced as being of relevance to the potential endoscopic surveillance of Barrett's oesophagus.

The success rate of current oesophageal endoscopy is so low as to be of questionable value. There is still a need for a feasible, reliable and cost-effective technique for detecting early stage Barrett's dysplasia before it has progressed to cancer of the oesophagus. Detection of dysplasia is the key step in patient management. Metaplasia is a much more common disease with lower risk than dysplasia and is easily detectable using white light. Ideally, this technique needs to avoid the sampling problems of prior art biopsy approaches, and to have good sensitivity for the detection of dysplastic lesions. Thus, the problem with current techniques is that a significant number of dysplastic lesions are missed and the majority of the tissue is not examined due to the random sampling procedure. The improved technique would then permit the monitoring of patients with Barrett's metaplasia periodically to detect any transition to precancer (dysplasia) or to oesophageal cancer at the earliest possible stage.

### THE PRESENT INVENTION

The present invention provides contrast agents for optical imaging of oesophageal cancer and Barrett's oesophagus in patients. The contrast agents are based on esters of 5-amino levulinic acid (5-ALA). A preferred such ester is Hexvix™ (hexaminolevulinate). The contrast agents may be used in the diagnosis of oesophageal cancer and Barrett's oesophagus, for follow up of progress in disease development, and for follow up of treatment of oesophageal cancer and Barrett's oesophagus. The agents are delivered as particular pharmaceutical compositions and by particular routes of administration.

Hexvix™ (hexaminolevulinate) is licensed for the detection of bladder cancer using fluorescence endoscopy. Hexvix acts by increasing the activity of the heme biosynthesis pathway, resulting in higher levels of fluorescent protoporphyrin IX (PPIX) in the target cells.

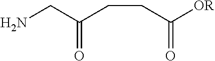

R═H, 5-ALA
R=n-hexyl, hexylaminolevulinate (HAL) or Hexvix™
R=methyl, Metvix™

Cystoscopy with Hexvix increases sensitivity of detecting bladder carcinoma in situ to 97% as opposed to 58% with white light cystoscopy [Frampton et al., Cancer Drugs, 66, 571-578 (2006)].

The 5-ALA esters are more lipophilic than 5-ALA, and exhibit greater accumulation in the urothelium compared to 5-ALA [Marti et al, J. Urol., 162, 546-552 (1999)]. The 5-ALA esters are therefore likely to show increased uptake in oesophageal tissues, permitting the use of lower concentrations of ALA derivative in contact with the tissue, without loss of the high sensitivity of detection. The lower doses are anticipated to improve the safety margin, aspects and potentially reduce tissue incubation time.

### DETAILED DESCRIPTION OF THE INVENTION

In a first aspect, the present invention provides a method of data acquisition useful in the diagnosis of a disease state of the oesophagus of the mammalian body in vivo, said method comprising:
(i) administration of a pharmaceutical composition comprising an ALA-ester of Formula (I) or a pharmaceutically acceptable salt thereof to a mammalian subject:

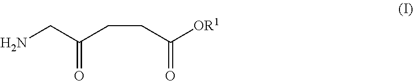

where:
R¹ is C₁₋₁₀ alkyl, C₁₋₁₀ alkoxyalkyl, C₁₋₁₀ hydroxyalkyl, C₁₋₁₀ fluoroalkyl or —C₃₋₆aryl(C₁₋₈ alkyl);
(ii) waiting for a period of time post-administration to allow:
   (a) the ALA-ester from step (i) to accumulate at one or more areas of interest within the oesophagus; and/or
   (b) the ALA-ester from step (i) to be converted to protoporphyrin IX;
(iii) illuminating said area of interest with an excitation light;
(iv) detecting the light generated by fluorescence of the protoporphyrin IX from step (ii)(b), using a fluorescence detector;
wherein the administration of step (i) is carried out by one or more of the following routes:
   (A) oral administration of a composition further comprising sodium alginate;
   (B) oral administration of a lozenge which comprises the composition;
   (C) spraying of the composition as an aerosol onto the walls of the oesophagus;
   (D) direct contact of the composition as an aqueous, non-hydrogel solution with the walls of the oesophagus.

The waiting time of step (ii), varies with the route of administration and the particular ester used. Hexyl esters have shown to have better penetration rates than other esters [Marti et al, J. Urol. 162, 546-552 (1999)]. The local administration route necessitates less time between administration and imaging (ca. 1-2 hours), versus 3-6 hours for the oral administration route. The optimum waiting times of steps (ii)(a) and (ii)(b) are best determined empirically within the above guidelines for the particular 5-ALA ester of interest.

The illuminating step (iii) and the detecting step (iv) use optical imaging techniques. By the term “optical imaging” is meant any method that forms an image for detection, staging or diagnosis of disease, follow up of disease development or for follow up of disease treatment based on interaction with light in the red to near-infrared region (wavelength 600-1200 nm). In the present application, the preferred illumination wavelength range is in the blue region (around 350-480 nm; maximum absorption at 405 nm), inducing fluorescence in the red region (600-750 nm; maxima at 636 and 703 nm). Optical imaging further includes all methods from direct visualization without use of any device and involving use of devices such as various scopes, catheters and optical imaging equipment, eg. computer-assisted hardware for tomographic presentations. The modalities and measurement techniques include, but are not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarization, luminescence, fluorescence lifetime, quantum yield, and quenching. Further details of these techniques are provided by: (Tuan Vo-Dinh (editor): “Biomedical Photonics Handbook” (2003), CRC Press LCC; Mycek & Pogue (editors): “Handbook of Biomedical Fluorescence” (2003), Marcel Dekker, Inc.; Splinter & Hopper: “An Introduction to Biomedical Optics” (2007), CRC Press LCC.

Suitable alkyl or alkoxyalkyl R¹ groups may be straight chain or branched. The ALA-ester may suitably be supplied in free base or salt form as a pharmaceutically acceptable salt. Suitable such salts are acid addition salts with physiologically acceptable organic or inorganic acids. Suitable acids include, for example, hydrochloric, hydrobromic, sulphuric, phosphoric, acetic, lactic, citric, tartaric, succinic, maleic, fumaric and ascorbic acids. Procedures for salt formation are conventional in the art.

The ALA-ester of Formula I is administered as a pharmaceutical composition, said pharmaceutical composition comprising the ALA-ester or pharmaceutically acceptable salt thereof, together with a biocompatible carrier in a form suitable for mammalian administration. The “biocompatible carrier” is a fluid, especially a liquid, in which the ALA-ester can be suspended or dissolved, preferably dissolved, such that the composition is physiologically tolerable, ie. can be administered to the mammalian body without toxicity or undue discomfort. The biocompatible carrier is suitably a carrier liquid such as sterile, pyrogen-free water for injection; an aqueous solution such as saline (which may advantageously be balanced so that the final product for administration is isotonic); an aqueous solution of one or more tonicity-adjusting substances (eg. salts of plasma cations with biocompatible counterions), sugars (e.g. glucose or sucrose), sugar alcohols (eg. sorbitol or mannitol), glycols (eg. glycerol), or other non-ionic polyol materials (eg. polyethyleneglycols, propylene glycols and the like). Preferably the biocompatible carrier is pyrogen-free water for injection or isotonic saline.

The ALA-ester or pharmaceutically acceptable salt thereof and biocompatible carrier are each supplied in suitable vials or vessels which comprise a sealed container which permits maintenance of sterile integrity and/or radioactive safety, plus optionally an inert headspace gas (eg. nitrogen or argon), whilst permitting addition and withdrawal of solutions by syringe or cannula. A preferred such container is a septum-sealed vial, wherein the gas-tight closure is crimped on with an overseal (typically of aluminium). The closure is suitable for single or multiple puncturing with a hypodermic needle (e.g. a crimped-on septum seal closure) whilst maintaining sterile integrity. Such containers have the additional advantage that the closure can withstand vacuum if desired (eg. to change the headspace gas or degas solutions), and withstand pressure changes such as reductions in pressure without permitting ingress of external atmospheric gases, such as oxygen or water vapour.

Preferred multiple dose containers comprise a single bulk vial (e.g. of 10 to 60 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, or “unit dose” and are therefore preferably a disposable or other syringe suitable for clinical use. The pharmaceutical compositions of the present invention preferably have a dosage suitable for a single patient and are provided in a suitable syringe or container, as described above.

Suitable concentrations of the ALA-ester in the pharmaceutical composition are in the range 0.01 to 0.5%, preferably 0.05 to 0.3%, most preferably 0.1 to 0.2%. When the ALA-ester is n-hexyl (Hexvix), an especially preferred range is 0.15 to 0.2%, particularly 0.16-0.18%

The pharmaceutical composition may optionally contain additional bio compatible excipients such as an antimicrobial preservative, pH-adjusting agent, filler, stabiliser, chelating agent or osmolality adjusting agent. By the term “antimicrobial preservative” is meant an agent which inhibits the growth of potentially harmful micro-organisms such as bacteria, yeasts or moulds. The antimicrobial preservative may also exhibit some bactericidal properties, depending on the dosage employed. The main role of the antimicrobial preservative(s) of the present invention is to inhibit the growth of any such micro-organism in the pharmaceutical composition. The antimicrobial preservative may, however, also optionally be used to inhibit the growth of potentially harmful micro-organisms in one or more components of kits used to prepare said composition prior to administration. Suitable antimicrobial preservative(s) include: the parabens, ie. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the composition is within acceptable limits (approximately pH 4.0 to 10.5) for human or mammalian administration. Suitable such pH-adjusting agents include pharmaceutically acceptable buffers, such as tricine, phosphate or TRIS [ie. tris(hydroxymethyl)aminomethane], and pharmaceutically acceptable bases such as sodium carbonate, sodium bicarbonate or mixtures thereof. When the composition is employed in kit form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

The term chelating agent has its conventional meaning. One or more chelating agents may beneficially be included in the ALA-ester pharmaceutical composition in order to enhance accumulation of PPIX. The chelation of iron by the chelating agent prevents its incorporation into PPIX to form haem by the action of the enzyme ferrochelatase, thereby leading to a build-up of PPIX. The imaging contrast is thus enhanced.

Aminopolycarboxylic acid chelating agents are particularly suitable in this regard, including any of the chelants described in the literature for metal detoxification or for the chelation of paramagnetic metal ions in magnetic resonance imaging contrast agents. Particular mention may be made of EDTA, CDTA (cyclohexane diamine tetraacetic acid), DTPA and DOTA. EDTA is preferred. To achieve the desired iron-chelation, desferrioxamine and other biocompatible siderophores may also be used, e.g. in conjunction with aminopolycarboxylic acid chelating agents such as EDTA. The chelating agent may conveniently be used at a concentration of 1 to 20% eg. 2 to 10% (w/w).

With oral administration of 5-ALA, PPIX formation appears to be higher in mucosal epithelia, including urothelium and the GI tract. The main drawback of oral administration is photosensitivity, with patients needing to avoid exposure to light for several hours post administration. For 5-ALA, local administration resulted in relatively lower levels of fluorescence than systemic application. This could be due to less efficient tissue penetration of topically applied 5-ALA. Local administration requires dual endoscopy (for both administration and imaging). That, and the need for longer patient tranquilisation and/or use of a local anaesthetic make this approach less favourable than oral administration from a clinical practice point of view.

Administration route (A) involves sodium alginate compositions. Sodium alginate is a polysaccharide isolated from seaweed. Such alginate compositions are known in the art for drug delivery [Coviello et al, Expert Opin. Drug Deliv., 3, 395-404 (2006); Tonnesen et al, Drug Dev. Ind. Pharm., 28, 621-630 (2002)]. Sodium alginate has successfully been used for oesophageal drug delivery (e.g. Gaviscon™), which coats the lower oesophagus to protect against acid exposure. Polysaccharide macromolecules have the advantages over synthetic polymers that they are widely present in living organisms, and non-toxic, biocompatible and can be obtained from renewable sources. Batchelor et al [Eur. J. Pharmaceut. Biopharm., 57, 295-298 (2004)] have published on the feasibility of alginate formulations for the delivery of model drug particles to the oesophagus.

Administration route (B) involves a lozenge which comprises the composition. In this route, the 5-ALA ester is formulated similarly to a throat lozenge or pastille which slowly dissolves in the patient's mouth, and the released ester then migrates slowly down the oesophagus over a period of time. Examples of this approach are provided by Shaoul et al [Aliment. Pharmacol. Ther., 24(4), 687-694 (2006)] and On et al [ibid, 15(9), 1385-1388 (2001)]. Lozenges have the advantages of easier application (less discomfort for patient), plus a prolonged period of application (which may help optimise signal intensity).

Administration route (C) involves spraying of the composition as an aerosol onto the walls of the oesophagus. The aerosol particles preferably have a particle size such that the particles do not enter the lung circulation after patient administration in vivo (generally >10 microns). Powder-based aerosols are expected to improve contact with the surface of the oesophagus, via the powder dissolving into mucus. Whilst there may be a risk of reduced aerosol delivery to the lower part of the oesophagus, peristaltic motion from oesophageal muscles should assist in distributing the agent further down the oesophagus.

Administration route (D) involves direct contact of the composition as an aqueous, non-hydrogel solution with the walls of the oesophagus. In that case, the ALA-ester is delivered topically as a conventional aqueous solution.

### Preferred Aspects.

The disease state of the oesophagus is preferably Barrett's oesophagus, most preferably dysplasia of the oesophagus.

The mammalian body of the first aspect is preferably the human body, most preferably the intact human body (ie. without need for intraoperative techniques).

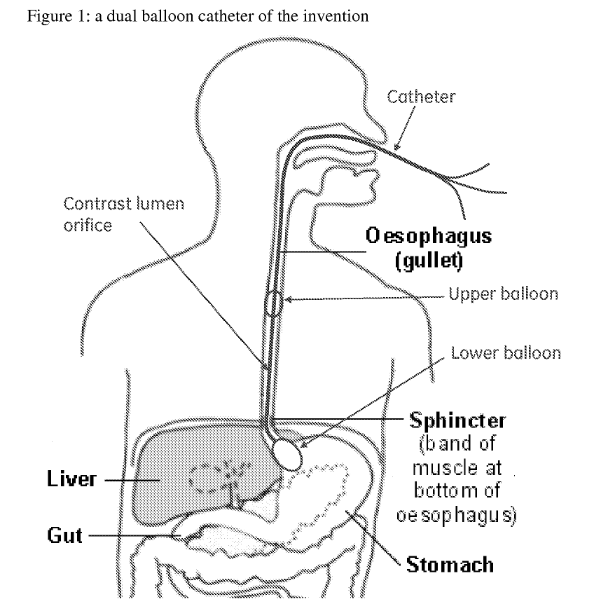

A preferred method of topical administration for route (D) is via use of a balloon catheter, especially a dual balloon catheter, to permit local administration inside the oesophagus. FIG. 1 and Example 2 illustrate the use of such a catheter. Suitable dual balloon catheters consist of a disposable thin rubber catheter with a large inflatable balloon at the tip, and another smaller one some 10-15 cm from the tip. The balloons can be inflated via separate channels. A third lumen for instillation of fluids has its orifice in the section between the balloons. The composition is suitably not a hydrogel, so that the viscosity of the solution is low enough to facilitate transport within the catheter.

Sengstaken tubes are known in the art and have similarities with the dual balloon catheter. They are used in the oesophagus in a similar manner (being swallowed, then inflated and left in place for some time). Sengstaken tubes, however, have only one balloon, and no lumen for injection of drug substances. Dual balloon catheters with similar arrangements of balloons are used in the management of severe nose bleeds, but these catheters lack the channel for injecting drug substances between the balloons, and are probably too small for use in the oesophagus.

The presence of the upper balloon of the invention will induce peristaltic movements in the oesophagus, which ensures mixing and optimal homogeneous contact between the contrast agent and the mucosa. Such dual balloon catheters have been used in the clinic for treatment of acute life-threatening oesophageal bleeding—so called “balloon tamponade” [Pasquale, Crit. Care Clin., 8(4), 743-753 (1992); Goff Gastroeneterol. Clin. N. Amer., 22(4) 779-800 (1993); Christensen Nursing Crit. Care, 9(2), 58-63 (2004)].

The main advantage of the dual balloon administration route compared to oral administration is that the ALA-ester stays in the oesophagus for some time without being transported down to the stomach by peristaltic motion. An adequate contact incubation time with the mucosa can thus be achieved at a fixed, controlled concentration over a fully-flexible contact time. The contact time can thus be optimised, if appropriate, for an individual patient.

In Formula I, R¹ is preferably C₁₋₈ alkyl, C₁₋₈ alkoxyalkyl or —C₄₋₆aryl(C₁₋₆ alkyl). When R¹ is C₁₋₈ alkyl, preferred straight chain alkyl groups are methyl or hexyl. Preferred branched chain alkyl groups are straight chain C₂₋₆ alkyl groups substituted by one or more C₁₋₂ alkyl groups. When R¹ is C₁₋₈ alkoxyalkyl, preferred such groups comprise 2 or 3 ether oxygen atoms. Most preferred such groups are —CH₂CH₂OCH₂CH₂OEt or —CH₂CH₂OCH₂CH₂OCH₂CH₂OMe. When R¹ is —C₄₋₆aryl(C₁₋₆ alkyl), a preferred such group is benzyl. A most preferred R¹ group is n-hexyl.

A preferred such salt of the ALA-ester is the hydrochloride salt.

The pharmaceutical compositions may be prepared under aseptic manufacture (ie. clean room) conditions to give the desired sterile, non-pyrogenic product. It is preferred that the key components, especially the associated reagents plus those parts of the apparatus which come into contact with the imaging agent (eg. vials) are sterile. The components and reagents can be sterilised by methods known in the art, including: sterile filtration, terminal sterilisation using e.g. gamma-irradiation, autoclaving, dry heat or chemical treatment (e.g. with ethylene oxide). It is preferred to sterilise some components in advance, so that the minimum number of manipulations needs to be carried out. As a precaution, however, it is preferred to include at least a sterile filtration step as the final step in the preparation of the pharmaceutical composition.

The pharmaceutical composition of the may preferably be prepared from a kit. Such kits suitably comprise the ALA-ester of Formula I, or pharmaceutically acceptable salt thereof, in sterile, solid form such that, upon reconstitution with a sterile supply of the biocompatible carrier, dissolution occurs to give the desired pharmaceutical composition. In such kits, the ALA-ester is preferably employed as a pharmaceutically acceptable salt, since that provides a solid form amenable to purification or pharmaceutical grade. A preferred sterile, solid form of the ALA-ester is a lyophilised solid. The sterile, solid form is preferably supplied in a pharmaceutical grade container, as described for the pharmaceutical composition (above). When the kit is lyophilised, the formulation may optionally comprise a cryoprotectant chosen from a saccharide, preferably mannitol, maltose or tricine. The lyophilised ALA-ester, plus other optional excipients as described above, is preferably provided as a powder in a suitable vial or container. The agent is then designed to be reconstituted with the desired biocompatible carrier to the pharmaceutical composition in a sterile, a pyrogenic form which is ready for mammalian administration.

The data acquisition of the first aspect is preferably used to construct an image of one or more areas of interest of the oesophagus of the mammalian subject.

The illumination light is preferably of wavelength 350-480 nm. The optical imaging method is preferably fluorescence endoscopy. A preferred optical imaging method of the first aspect is Fluorescence Reflectance Imaging (FRI). In FRI, the ALA-ester of the present invention is administered to a subject to be diagnosed, and subsequently [step (iii)] a tissue surface of the subject is illuminated with an excitation light—usually continuous wave (CW) excitation. The light excites the PPIX formed from the ALA-ester, as described above. Fluorescence from the PPIX, which is generated by the excitation light, is detected using a fluorescence detector [step (iv)]. The returning light is preferably filtered to separate out the fluorescence component (solely or partially). An image is formed from the fluorescent light. Usually minimal processing is performed (no processor to compute optical parameters such as lifetime, quantum yield etc.) and the image maps the fluorescence intensity. The higher levels of the PPIX in the diseased area, producing higher fluorescence intensity. Thus, the disease area produces positive contrast in a fluorescence intensity image. The image is preferably obtained using a CCD (charge-coupled device) camera or chip, such that real-time imaging is possible.

The apparatus for generating the excitation light of step (iii) may be a conventional excitation light source such as: a laser (e.g., ion laser, dye laser or semiconductor laser); halogen light source or xenon light source. Various optical filters may optionally be used to obtain the optimal excitation wavelength.

A preferred FRI method comprises the steps as follows:
(a) the excitation light of step (iii) is from a light source with a pre-determined time varying intensity to excite the protoporphyrin IX, the tissue in the area of interest multiply-scattering the excitation light;
(b) detecting a multiply-scattered light emission from the tissue in response to said exposing;
(c) quantifying a fluorescence characteristic throughout the tissue from the emission by establishing a number of values with a processor, the values each corresponding to a level of the fluorescence characteristic at a different position within the tissue, the level of the fluorescence characteristic varying with heterogeneous composition of the tissue; and
(d) generating an image of the tissue by mapping the heterogeneous composition of the tissue in accordance with the values of step (c).

In step (i), the excitation light is preferably continuous wave (CW) in nature. In step (iii), the light detected is preferably filtered. An especially preferred FRI method is fluorescence endoscopy, as described by Stepinac et al [Endoscopy, 35(8), 663-668 (2003)].

An alternative imaging method of the first aspect uses FDPM (frequency-domain photon migration). This has advantages over continuous-wave (CW) methods where greater depth of detection of the imaging agent within tissue is important [Sevick-Muraca et al, Curr. Opin. Chem. Biol., 6, 642-650 (2002)]. For such frequency/time domain imaging, it is advantageous if the fluorescent properties can be modulated depending on the tissue depth of the lesion to be imaged, and the type of instrumentation employed.

The FDPM method is as follows:
(a) exposing light-scattering biological tissue of said mammalian body having a heterogeneous composition to light from a light source with a pre-determined time varying intensity to excite the PPIX, the tissue multiply-scattering the excitation light;
(b) detecting a multiply-scattered light emission from the tissue in response to said exposing;
(c) quantifying a fluorescence characteristic throughout the tissue from the emission by establishing a number of values with a processor, the values each corresponding to a level of the fluorescence characteristic at a different position within the tissue, the level of the fluorescence characteristic varying with heterogeneous composition of the tissue; and
(d) generating an image of the tissue by mapping the heterogeneous composition of the tissue in accordance with the values of step (c).

The fluorescence characteristic of step (c) preferably corresponds to levels of PPIX, and preferably further comprises mapping a number of quantities corresponding to adsorption and scattering coefficients of the tissue before administration of the ALA-ester. The fluorescence characteristic of step (c) preferably corresponds to at least one of fluorescence lifetime, fluorescence quantum efficiency or fluorescence yield. The fluorescence characteristic is preferably independent of the intensity of the emission and independent of ALA-ester concentration in tissue.

The quantifying of step (c) preferably comprises: (i) establishing an estimate of the values, (ii) determining a calculated emission as a function of the estimate, (iii) comparing the calculated emission to the emission of said detecting to determine an error, (iv) providing a modified estimate of the fluorescence characteristic as a function of the error. The quantifying preferably comprises determining the values from a mathematical relationship modelling multiple light-scattering behaviour of the tissue. The method of the first option preferably further comprises monitoring a metabolic property of the tissue in vivo by detecting variation of said fluorescence characteristic.

5-Amino levulinic acid, as the hydrochloride salt and the corresponding methyl ester of 5-ALA are commercially available from Sigma-Aldrich. The synthesis of the hexyl ester of 5-ALA is given in Example 1.

In a second aspect, the present invention provides a method of in vivo imaging of a disease state of the oesophagus of the mammalian body, wherein said method comprises:
(i) provision of a mammalian subject to whom had been previously administered the ALA-ester of Formula (I) as defined in the first aspect via one of the administration routes (A)-(D) of in the first aspect;
provision of a mammalian subject to whom had been previously administered an ALA-ester of Formula (I) or a pharmaceutically acceptable salt thereof, or a pharmaceutical composition thereof as described;
(ii) waiting for a period of time to allow:
   (a) the ALA-ester from step (i) to accumulate at one or more areas of interest within the oesophagus; and/or
   (b) the ALA-ester from step (i) to be converted to protoporphyrin IX;
(iii) illuminating said area of interest with an excitation light;
(iv) detecting the light generated by fluorescence of the protoporphyrin IX from step (ii)(b), using a fluorescence detector;
(v) optionally filtering the light detected by the fluorescence detector to separate out the fluorescence component;
(vi) converting the fluorescence signals detected in steps (iv) or (v) into one or more images of said area of interest.

Steps (ii)-(vi) and preferred embodiments thereof are as described in the first aspect (above). By “previously administered” in Step (i) is meant that the step involving the clinician, wherein the ALA-ester is given to the patient has already been carried out prior to imaging, preferably at a known time before the imaging method of the second aspect.

In a third aspect, the present invention provides the use of the ALA-ester, or pharmaceutically acceptable salt thereof, of Formula I as defined in the first aspect in the manufacture of a composition for use in the method of data acquisition of the first aspect or the method of imaging of the second aspect.

Preferred aspects of the ALA-ester and its salts for use in the third aspect are as defined above.

In a fourth aspect, the present invention provides the use of a kit for the preparation of a pharmaceutical composition of an ALA-ester or pharmaceutically acceptable salt thereof of Formula I, said kit comprising separate containers each comprising:
(i) the ALA-ester of Formula I as defined in the first aspect;
(ii) a supply of biocompatible carrier;
in the method of data acquisition of the first aspect or the method of imaging of the second aspect.

The biocompatible carrier, and preferred aspect thereof are as described in the first aspect b(above). Preferred aspects of the ALA-ester, its salts kits for the preparation of pharmaceutical compositions of the ALA-ester for use in the fourth aspect are as defined above.

In a fifth aspect, the present invention provides the use of a dual balloon catheter in the method of the first aspect, where the ALA-ester of Formula I is administered via route (D).

In a sixth aspect, the present invention provides a method of detection, staging, diagnosis, monitoring of disease progression or monitoring of treatment of a disease state of the oesophagus of the mammalian body which comprises either:
(i) the method of data acquisition of the first aspect; or
(ii) the method of imaging of the second aspect.

Preferred aspects of the method of data acquisition and the method of imaging of the sixth aspect are as described in the first and second aspects (above) respectively. The disease state of the sixth aspect oesophagus is preferably Barrett's oesophagus or dysplasia. The method of the sixth aspect is preferably used in biopsy guidance.

The invention is illustrated by the non-limiting Examples detailed below. Example 1 provides the synthesis of the hexyl ester of 5-ALA. Example 2 is a prophetic Example, describing how the oesophagus ALA-ester administration would be carried out using a dual balloon catheter technique. Example 3 is a prophetic Example, describing how the endoscopic evaluation of the invention would be carried out.

### Example 1

### Preparation of n-Hexyl 5-Aminolevulinic Hydrochloride (ALA Hexylester)

5-Aminolevulinic acid hydrochloride (Sigma, 2.0 g) was dissolved in dry n-hexanol (25 g) containing 5-6 drops of concentrated hydrochloric acid in a 50 ml glass reactor equipped with a reflux condenser and a thermometer. The reaction mixture was heated at 50-60° C. with stirring for approximately 3 days. The excess n-hexanol was then removed in vacuo and the product finally dried under high vacuum, giving n-hexyl 5-aminolevulinate hydrochloride (2.4 g). The structure was confirmed by ¹H-NMR spectroscopy in d₆-DMSO.

### Example 2

### Use of Dual Balloon Catheter in Oesophagus Administration Prior to Imaging (Prophetic Example)

FIG. 1 illustrates the suggested use of such a catheter. Such dual balloon catheters consist of a disposable thin rubber catheter with a large inflatable balloon at the tip, and another smaller one some 10-15 cm from the tip. The balloons can be inflated via separate channels. A third lumen for instillation of fluids has its orifice in the section between the balloons. The catheter would be introduced via the nose of the mammalian subject after local application of a suitable local anaesthetic to the nasal and pharyngeal mucosa. Swallowing of such a catheter is not expected to be particularly “invasive”, since after application of a local anaesthetic spray the catheter would be easily introduced via the nose with little patient discomfort.

The catheter would be swallowed, and advanced so that the tip entered the gastric cavity. The balloon at the tip would then be inflated to a diameter that prevents retraction into the oesophagus, and a slight tension would be applied to the catheter in order to make the balloon create a seal between the oesophagus and the gastric cavity. The upper balloon would then be inflated to seal off the upper part of the oesophagus and prevent fluids entering from above. The ALA-ester would then be instilled via the fluid lumen, and the catheter left in place for the required time interval. The remaining ALA-ester agent might then be aspirated, saline might be used for washing off excess ester from the mucosa, the balloons are deflated and the catheter would be removed before carrying out the endoscopic examination.

The presence of the upper balloon will induce peristaltic movements in the oesophagus, which will ensure mixing and optimal homogeneous contact between the ALA-ester agent and the mucosa.

### Example 3

### Endoscopic Examination Following Administration of 5-ALA Ester (Prophetic Example)

Administration the 5-ALA ester will be followed by a waiting period of appropriate duration (in the range of 30 min to 5 hours). Endoscopy will then be performed using a standard flexible endoscope connected to a light source delivering white or blue light, emitting blue light at a wavelength of 350-480 nm. The endoscope will be attached to a camera with an imaging processing system displaying real time fluorescence data on a video screen. During endoscopy it will be possible to switch between the white and blue mode. The areas containing high PPIX concentrations will appear with characteristic red fluorescence under blue light illumination. Biopsies will be taken using red fluorescence as a guidance for higher risk areas within Barrett's oesophagus segments.

What is claimed is:

1. A method of data acquisition useful in the diagnosis of a disease state of the oesophagus of the mammalian body in vivo, said method comprising:
(i) administration of a pharmaceutical composition comprising an ALA-ester of Formula (I) or a pharmaceutically acceptable salt thereof to a mammalian subject: 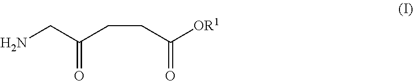
where:
R¹ is C₁₋₁₀ alkyl, C₁₋₁₀ alkoxyalkyl, C₁₋₁₀ hydroxyalkyl, C₁₋₁₀ fluoroalkyl or —C₃₋₆aryl(C₁₋₈ alkyl);
(ii) waiting for a period of time post-administration to allow:
(a) the ALA-ester from step (i) to accumulate at one or more areas of interest within the oesophagus; and/or
(b) the ALA-ester from step (i) to be converted to protoporphyrin IX;
(iii) illuminating said area of interest with an excitation light;
(iv) detecting the light generated by fluorescence of the protoporphyrin IX from step (ii)(b), using a fluorescence detector;
wherein the administration of step (i) is carried out by one or more of the following routes:
(A) oral administration of a composition further comprising sodium alginate;
(B) oral administration of a lozenge which comprises the composition;
(C) spraying of the composition as an aerosol onto the walls of the oesophagus;
(D) direct contact of the composition as an aqueous, non-hydrogel solution with the walls of the oesophagus.

2. The method of claim 1, where R¹ is C₁₋₈ alkyl, C₁₋₈ alkoxyalkyl or —C₄₋₆aryl(C₁₋₆ alkyl).

3. The method of claim 2, where R¹ is n-hexyl.

4. The method of claim 1, where administration route (A) is used.

5. The method of claim 1, where administration route (D) is used, and said direct contact is achieved via endoscopy or by use of a balloon catheter.

6. The method of claim 5, where said balloon catheter is a dual balloon catheter used to contact the ALA-ester pharmaceutical composition with the walls of the oesophagus for a period of time sufficient to permit uptake of the ALA-ester into oesophageal tissue.

7. The method of claim 1, where the pharmaceutical composition is prepared from a kit, said kit comprising separate containers as follows:
(i) the ALA-ester of Formula (I) or pharmaceutically acceptable salt thereof as defined in claim 1; and
(ii) a supply of biocompatible carrier.

8. The method of claim 1, where the disease state is oesophageal cancer or Barrett's oesophagus.

9. The method of claim 1, where the disease state is dysplasia.

10. The method of claim 1, where the data acquisition is used to construct an image of one or more areas of interest of the oesophagus of the mammalian subject.

11. A method of in vivo imaging of a disease state of the oesophagus of the mammalian body, wherein said method comprises:
(i) provision of a mammalian subject to whom had been previously administered the ALA-ester of Formula (I) as defined in any one of claim 1 via one of the administration routes (A)-(D) of claim 1;
(ii) waiting for a period of time to allow:
(a) the ALA-ester from step (i) to accumulate at one or more areas of interest within the oesophagus; and/or
(b) the ALA-ester from step (i) to be converted to protoporphyrin IX;
(iii) illuminating said area of interest with an excitation light;
(iv) detecting the light generated by fluorescence of the protoporphyrin IX from step (ii)(b), using a fluorescence detector;
(v) optionally filtering the light detected by the fluorescence detector to separate out the fluorescence component;
(vi) converting the fluorescence signals detected in steps (iv) or (v) into one or more images of said area of interest.

12. The method of claim 11, where the excitation light of step (iii) is continuous wave (CW) in nature.

13. The method of claim 11, wherein:
(a) the excitation light of step (iii) is from a light source with a pre-determined time varying intensity to excite the protoporphyrin IX, the tissue in the area of interest multiply-scattering the excitation light;
and further comprising:
(b) detecting a multiply-scattered light emission from the tissue in response to said illuminating;
(c) quantifying a fluorescence characteristic throughout the tissue from the emission by establishing a number of values with a processor, the values each corresponding to a level of the fluorescence characteristic at a different position within the tissue, the level of the fluorescence characteristic varying with heterogeneous composition of the tissue; and
(d) generating an image of the tissue by mapping the heterogeneous composition of the tissue in accordance with the values of step (c).

14. The method of claim 11 wherein the method of in vivo imaging is an optical imaging method comprising fluorescence endoscopy.

15. The method of claim 11, where the disease state is oesophageal cancer, Barrett's oesophagus or dysplasia.

16-22. (canceled)
